(19) United States
(12) Patent Application Publication
(10) Pub. No.: US 2007/0134347 A1
(43) Pub. Date: Jun. 14, 2007

(54) METHOD OF MAKING AN EXTRACT OF BIOLOGICALLY ACTIVE ADDITIVE

(75) Inventors: Anatoly Anatolevich LISOVSKYl, Moskoskaya obl (RU); Boris Moiseevich KERSHENGOLTS, Moskoskaya obl (RU); Pavel Aleksandrovich REMIGAILO, g. Yakutsk (RU)

Correspondence Address: PATENTTM.US, P. O. BOX 82788, PORTLAND, OR, 97282-0788, US

(73) Assignee: Anatoly Anatolevich LISOVSKY, Moskoskaya obl. (RU)

(21) Appl. No.: 11/558,369

(22) Filed: Nov. 9, 2006

(30) Foreign Application Priority Data
Dec. 9, 2005 (RU) 2005138346

(51) Int. Cl. A61K 35/32

(52) U.S. Cl. 424/549

(57) ABSTRACT

This invention relates to the food industry and, in particular, to a technology of making an extract of a biologically active additive intended for wide application, and may be used as a biologically active food additive in pharmacology and the food industry. According to this invention, a method of making an extract of a biologically active additive consists in that second and third sort young reindeer antlers with their pelage for a length of 10-12 cm from the ends are taken, cleaned from dirt and washed with an alcohol solution, dried at 10-12° C. until their humidity is app. 30% (for 35 hours), the obtained raw material is ground down to a fraction of 3.7-4.69 microns, then the obtained powder is extracted with 50% ethyl alcohol at 17-25° C. for 15-27 hours under periodic mixing of the solution, after settling the precipitate is removed and once again extracted in a new solution of 50% ethyl alcohol at the same conditions, this process being repeated at least three times for the purpose of obtaining a first, second and third semi-finished extract according to the extraction stages, then the obtained semi-finished extracts are mixed together, ethanol and acetic acid are added for the purpose of obtaining 50% strength and pH=4.3-5.1, then the semi-finished extract is held at T=−49° C. (or 47-51° C.) for 8 hours, then it is filtered at 0-5° C. for obtaining the finished product in the form of an extract of a biologically active additive.

### FIELD OF THE INVENTION

This invention relates to the food industry and, in particular, to a technology of making an extract of a biologically active additive intended for wide application, and may be used as a biologically active food additive in pharmacology and the food industry.

### PRIOR ART

Among therapeutic and prophylactic agents there is a great number of vitamin-mineral complexes that are intended to cover a deficit in nutrition and organism strengthening as well as amino-acid preparations enriched with certain vitamins and microelements (see, RF Patent No. 2080871, A 61 K 38/01, publ. 1997, and RF Patent No. 2147808, A 23 D 9/00, publ. 1998).

Such food additives are intended to restore the vital protective functions of the human organism and are used directly at meals or are introduced into compositions of specialized foods for the purpose of enriching rations with certain nutrients and biologically active agents or their complexes.

Disadvantages of the said agents are: first, the presence of an alcohol which is unacceptable for persons inclined to alcoholism and is contraindicated for teenagers, and second, the form of such agents, i.e., tablets, is conservative, since it may not be used externally.

The closest technical solution is the invention described in Patent SU1822785, IPC A61 K 35/32, 1990. The subject of the invention is a method of making an extract from deer antlers, which includes grinding the source material, triple extracting by 50% ethyl alcohol at room temperature, mixing, joining the obtained extracts, incubating in cold, separating a precipitate, wherein the second and third sort young reindeer antlers with pelage are used as the source material, which is ground to powder state with subsequent filtration and incubation.

A disadvantage of the known method is low intensity of the production process, which is due to long-time incubation processes and low-quality filtration; in the result, the specific activity of the target product is lowered.

The objective of this invention is to significantly increase the specific activity of the finished product while simultaneously reducing the cost and time of its production.

In order to achieve the stated technical result, the inventive method of making a biologically active additive extract is carried out as follows: second and third sort young reindeer antlers with their pelage for a length of 10-12 cm from the ends are taken, cleaned from dirt and washed with an alcohol solution, then dried at 10-12° C. until their humidity is app. 30% (for 35 hours), the obtained raw material is ground down to a fraction of 3.7-4.69 microns, then the obtained powder is softly extracted with 50% ethyl alcohol at 17-25° C. for 15-27 hours under periodic mixing of the solution, after settling the precipitate is removed and once again extracted in a new solution of 50% ethyl alcohol at the same conditions, this process being repeated at least three times for the purpose of obtaining a first, second and third semi-finished extract according to the extraction stages, then the obtained semi-finished extracts are mixed together, ethanol and acetic acid are added for the purpose of obtaining 50% strength and pH=4.3-5.1, then the semi-finished extract is held at T=−49° C. (or 47-51° C.) for 8 hours, then it is filtered at 0-5° C. for obtaining the finished product in the form of an extract of a biologically active additive.

It is advisable to carry out filtration for achieving different purity degree in order to form specific useful properties of the obtained extract.

It is commonly known that organisms of plants, animals and human beings contain a complex of biologically active substances, this complex is considered as normal if it interacts with cells and target organs in a balanced way which is achieved by self-regulation of its all components as a whole system. Usually, when making biologically active additives and, on their basis, medicines, individual most active components are selected from the whole natural complex, which then become the basis for a new substance. There is, however, a great reason to believe that by maintaining the natural composition and ratio of active substances one may achieve a more pronounced and all-purpose effect manifested by a preparation in treating various diseases. The “Epsorin” preparation produced from young reindeer antlers by the proposed soft extraction technology comprises the composition of biologically active substances, which is close to the natural one, due to the fact that they are not lost in the production process contrary to other known technologies. It is known that the more severe is the habitat of an animal or plant, the more pronounced is the activity of all the organism adaptation systems and the more adaptogenic is the action of its complex of active substances. As the raw material for producing biologically active substances antlers are used, which are a hemopoietic organ adapted to the Arctic conditions; therefore, their derivative (extract) has a unique bio-potential.

According to this invention the method of making a biologically active additive extract comprises the following steps:

second and third sort young reindeer antlers with their pelage for a length of 10-12 cm from the ends are taken, it is just this length is determined by the maturity of an animal, development of its organism and a maximum quantity of useful substances;

antlers are cleaned from dirt and washed with an alcohol solution, dried at 10-12° C. until their humidity is app. 30% (for 35 hours) by a ventilation drying method, these steps being carried out promptly since antlers bleed after separation, their blood vessels are open, and an infection (dirt) may get into them;

the obtained raw material is ground down to a fraction of 3.7-4.69 microns; this grain size of the raw material is conditioned by the following: a fraction smaller than 3.7 microns gives a dust-like medium that is difficult in processing for technological reasons, the upper limit is determined by the efficiency of obtaining active substances (the range of grain size as well as the other parameters of the production process have been established experimentally);

the obtained raw material in the form of finely dispersed powder is softly extracted with 50% ethyl alcohol at 17-25° C. for 15-27 hours under periodic mixing of the solution, after settling the precipitate is removed and once again extracted in a new solution of 50% ethyl alcohol at the same conditions, this process being repeated at least three times for the purpose of obtaining a first, second and third semi-finished extract according to the extraction stages;

the obtained semi-finished extracts are mixed together, ethanol and acetic acid are added for the purpose of obtaining 50% strength and pH=4.3-5.1;

then the semi-finished extract is held at T=−49° C. (or 47-51° C.) for 8 hours, then it is filtered at 0-5° C. for obtaining the finished product in the form of an extract of a biologically active additive.

Filtration should be carried out for achieving different purity degree in order to form specific useful properties of an obtained extract.

The biological efficiency of the obtained additive is based on its content, due to the proposed technology, of: phospholipids, free amino acids, microelements, a balanced combination of water-soluble and fat-soluble vitamins, dibasic and tribasic organic acids as well as a combination of prostaglandins (intracellular regulators of action of many hormones) and peptides normalizing the reactivity of the immune system, a wide spectrum of anti-oxidants, biologically active derivatives of cholesterol (glucocorticosteroids and mineralocorticosteroids, sex hormones).

Moreover, the inventive extract of biologically active additive is not toxic within the concentration limits 10 times exceeding the recommended pharmaceutical doses and does not has a cumulative effect. One feature of the inventive extract is its high pharmacological activity in relation to antistress, adaptogenic, immunoregulatory, tonic, biostimulating properties as well as special properties.

An example of carrying out the inventive method of making a biologically active additive extract based on antlers of young reindeer.

Second and third sort young reindeer antlers with their pelage for a length of 11 cm from the ends are taken, cleaned from dirt and washed with a 30% alcohol solution. Clean and washed antlers with the pelage are dried by ventilation at 10° C. until their humidity is app. 30%, holding for 33 hours, in the result, the raw material is obtained, which is ground down to a fraction of 4.2 microns.

Then the obtained powder is softly extracted with 50% ethyl alcohol at 19° C. for 21 hours under periodic mixing of the solution, after settling the precipitate is removed and once again extracted in a new solution of 50% ethyl alcohol at the same conditions, this process being repeated at least three times for the purpose of obtaining a first, second and third semi-finished extract according to the extraction stages, then the obtained semi-finished extracts are mixed together, ethanol and acetic acid are added for the purpose of obtaining 50% strength and pH=4.7.

The obtained semi-finished extract is held at T=−49° C. for 8 hours, then it is filtered for the purpose of clarifying it at 0° C. until obtaining the finished product in the form of an extract of a biologically active additive.

This invention complies with the patentability criterion of “industrial applicability” since it may be realized with the use of existing means of production and known technologies. Application of this invention enables to solve the task of mass prophylaxis by immunocorrectors, hepatoprotectors and adaptogens.

What is claimed is:

1. A method of making an extract of a biologically active additive, comprising the following steps: taking second and third sort young reindeer antlers with their pelage for a length of 10-12 cm from the ends, cleaning them from dirt and washing with an alcohol solution, drying at 10-12° C. until their humidity is app. 30% (for 35 hours), grinding the obtained raw material down to a fraction of 3.7-4.69 microns, extracting the obtained powder with 50% ethyl alcohol at 17-25° C. for 15-27 hours under periodic mixing of the solution, removing the precipitate after settling and once again extracting it in a new solution of 50% ethyl alcohol at the same conditions, this process being repeated at least three times for the purpose of obtaining a first, second and third semi-finished extract according to the extraction stages, then mixing together the obtained semi-finished extracts, adding ethanol and acetic acid for the purpose of obtaining 50% strength and pH=4.3-5.1, then holding the semi-finished extract at T=−49° C. (or 47-51° C.) for 8 hours, and then filtering it at 0-5° C. for obtaining the finished product in the form of an extract of a biologically active additive.

2. A method according to claim 1, characterized in that the filtering step is carried out for obtaining different degrees of purity in order to ensure a possibility of forming specific useful properties of an obtained extract.
